(19) United States
(12) Patent Application Publication
(10) Pub. No.: US 2018/0078652 A1
(43) Pub. Date: Mar. 22, 2018

(54) NANOPORPHYRIN TELODENDRIMERS FOR TREATMENT OF VASCULAR ABNORMALITIES

(71) Applicant: THE REGENTS OF THE UNIVERSITY OF CALIFORNIA, Oakland, CA (US)

(72) Inventors: Hakan ORBAY, Oakland, CA (US); David SAHAR, Oakland, CA (US); Yuanpei LI, Oakland, CA (US); Kit S. LAM, Oakland, CA (US)

(21) Appl. No.: 15/568,749

(22) PCT Filed: Apr. 22, 2016
(86) PCT No.: PCT/US2016/029058

§ 371 Date: Oct. 23, 2017

Related U.S. Application Data
(60) Provisional application No. 62/152,656, filed on Apr. 24, 2015.

(51) Int. Cl. A61K 47/60; A61K 41/00; A61K 31/475; A61K 9/00; A61K 31/5377; A61K 47/54; A61N 5/06

(52) U.S. Cl. CPC A61K 47/60; A61K 41/0057; A61K 31/475; A61K 9/0019; A61N 2005/067; A61K 31/5377; A61K 47/554; A61N 5/062; A61K 9/0014

(57) ABSTRACT

Methods and compositions are provided for treating a vascular abnormality.

### CROSS-REFERENCES TO RELATED APPLICATIONS

This application claims priority to U.S. Provisional Application No. 62/152,656, filed Apr. 24, 2015, the contents of which are hereby incorporated by reference in the entirety for all purposes.

### BACKGROUND OF THE INVENTION

Infantile hemangioma (IH) is a vascular neoplasm affecting 10% of all children with significant morbidity and no effective treatment. As such, there is a need to develop therapeutics to treat IH and other vascular abnormalities.

### BRIEF SUMMARY OF THE INVENTION

In a first embodiment, the present invention provides a method of treating a vascular abnormality in a subject in need thereof by photodynamic or photothermal therapy, the method comprising: a) administering to the subject an effective amount of a photosensitizer; and b) exposing the vascular abnormality to an effective amount of electromagnetic radiation having a wavelength that is absorbed by the photosensitizer, thereby treating the vascular abnormality by photodynamic or photothermal therapy (or the combination thereof), wherein the photosensitizer comprises a compound of formula I: (B)ₖ-(PEG)ₘ-A(Y¹)ₚ-L¹-D-[Y²-L²-R]ₙ (I), wherein B is a binding ligand; each PEG is a polyethyleneglycol (PEG) polymer having a molecular weight of 1-100 kDa; A comprises at least one branched monomer unit X and is linked to at least one PEG group; D is a dendritic polymer having a single focal point group, a plurality of branched monomer units X and a plurality of end groups; each Y¹ and Y² is absent or a crosslinkable group independently selected from the group consisting of boronic acid, dihydroxybenzene and a thiol; each L¹ and L² is independently a bond or a linker, wherein L¹ is linked to the focal point group of the dendritic polymer; each R is independently selected from the group consisting of the end group of the dendritic polymer, a porphyrin, a hydrophobic group, a hydrophilic group, an amphiphilic compound and a drug, wherein at least one R group is a porphyrin; subscript k is 0 or 1; subscript m is an integer from 0 to 20; subscript n is an integer from 2 to 20, wherein subscript n is equal to the number of end groups on the dendritic polymer; and subscript p is from 0 to 8.

In a second embodiment, the present invention provides a composition comprising: a) a nanocarrier having an interior and an exterior, wherein the interior of the nanocarrier comprises a hydrophobic pocket; and b) an inhibitor of vascularization, wherein the inhibitor of vascularization is sequestered in the hydrophobic pocket of the nanocarrier, wherein the nanocarrier comprises a plurality of first conjugates wherein each conjugate comprises: a polyethylene glycol (PEG) polymer; at least two amphiphilic compounds having both a hydrophilic face and a hydrophobic face; at least one porphyrin; optionally at least two crosslinking groups; and a dendritic polymer covalently attached to the PEG, the amphiphilic compounds, the porphyrin and the crosslinking groups, wherein each conjugate self-assembles in an aqueous solvent to form the nanocarrier such that a hydrophobic pocket is formed in the interior of the nanocarrier by the orientation of the hydrophobic face of each amphiphilic compound towards each other, wherein the PEG of each conjugate self-assembles on the exterior of the nanocarrier, and wherein each conjugate is a compound of formula I: (B)ₖ-(PEG)ₘ-A(Y¹)ₚ-L¹-D-[Y²-L²-R]ₙ (I), wherein B is a binding ligand; each PEG is a polyethyleneglycol (PEG) polymer having a molecular weight of 1-100 kDa; A comprises at least one branched monomer unit X and is linked to at least one PEG group; D is a dendritic polymer having a single focal point group, a plurality of branched monomer units X and a plurality of end groups; each Y¹ and Y² is absent or a crosslinkable group independently selected from the group consisting of boronic acid, dihydroxybenzene and a thiol; each L¹ and L² is independently a bond or a linker, wherein L¹ is linked to the focal point group of the dendritic polymer; each R is independently selected from the group consisting of the end group of the dendritic polymer, a porphyrin, a hydrophobic group, a hydrophilic group, an amphiphilic compound and a drug, wherein at least one R group is a porphyrin; subscript k is 0 or 1; subscript m is an integer from 0 to 20; subscript n is an integer from 2 to 20, wherein subscript n is equal to the number of end groups on the dendritic polymer; and subscript p is from 0 to 8.

### BRIEF DESCRIPTION OF THE DRAWINGS

FIG. 1 illustrates an embodiment of phototherapy for treating infantile hemangioma (IH) in a mouse model.

FIG. 2A-2C illustrates disease progression in a mouse animal model of IH.

FIG. 3 illustrates a nanoporphyrin-(NP)based nanocarrier.

FIG. 4A-4C illustrates accumulation of a ⁶⁴Cu-loaded NP in an IH of a mouse as measured by PET.

FIG. 5 illustrates the results of an ex vivo biodistribution study of NP accumulation.

FIG. 6 illustrates accumulation of a dye-loaded NP in an IH of a mouse, and tissue distribution thereof, as measured by near infrared imaging (NIRFI).

FIG. 7 illustrates a scheme for studying efficacy of treatment of IH in a mouse model using an NP and phototherapy.

FIG. 8 illustrates results of the study depicted in FIG. 7.

FIG. 9 illustrates additional study groups using a NP loaded with an inhibitor of vascularization (e.g., propanolol) and/or targeted to IH cells via a targeting ligand (e.g., anti-CD133 antibody).

### DEFINITIONS

As used herein, the terms “dendrimer” and “dendritic polymer” refer to branched polymers containing a focal point, a plurality of branched monomer units, and a plurality of end groups. The monomers are linked together to form arms (or “dendrons”) extending from the focal point and terminating at the end groups. The focal point of the dendrimer can be attached to other segments of the compounds of the invention, and the end groups may be further functionalized with additional chemical moieties.

As used herein, the term “telodendrimer” refers to a dendrimer containing a hydrophilic PEG segment and one or more chemical moieties covalently bonded to one or more end groups of the dendrimer. These moieties can include, but are not limited to, hydrophobic groups, hydrophilic groups, amphiphilic compounds, and drugs. Different moieties may be selectively installed at a desired end groups using orthogonal protecting group strategies. Telodendrimers such as porphyrin containing telodendrimers, nanocarriers comprising such telodendrimers, including nanocarriers containing a drug (e.g., an inhibitor of vascularization), formulations containing such telodendrimers and/or nanocarriers, and methods of their making and use include those composition and methods described in international application publication WO 2014/093675; Lin et al., and Nat. Comm., 2014:5: 4712, which are hereby incorporated herein by reference in the entirety for any and all purposes.

As used herein, the term “nanocarrier” refers to a micelle resulting from aggregation of the dendrimer conjugates of the invention. The nanocarrier has a hydrophobic core and a hydrophilic exterior. The nanocarrier can be loaded with an imaging agent (e.g., hydrophobic fluorophore), a drug (e.g., an inhibitor of vascularization), or a combination thereof.

As used herein, the terms “monomer” and “monomer unit” refer to a diamino carboxylic acid, a dihydroxy carboxylic acid and a hydroxyl amino carboxylic acid. Examples of diamino carboxylic acid groups of the present invention include, but are not limited to, 2,3-diamino propanoic acid, 24-diaminobutanoic acid, 2,5-diaminopentanoic acid (omithine), 2,6-diaminohexanoic acid (lysine), (2-Aminoethyl)-cysteine, 3-amino-2-aminomethyl propanoic acid, 3-amino-2-aminomethyl-2-methyl propanoic acid, 4-amino-2-(2-aminoethyl) butyric acid and 5-amino-2-(3-aminopropyl) pentanoic acid. Examples of dihydroxy carboxylic acid groups of the present invention include, but are not limited to, glyceric acid, 2,4-dihydroxybutyric acid, glyceric acid, 2,4-dihydroxybutyric acid, 2,2-Bis(hydroxymethyl)propionic acid and 2,2-Bis(hydroxymethyl)butyric acid. Examples of hydroxyl amino carboxylic acids include, but are not limited to, serine and homoserine. One of skill in the art will appreciate that other monomer units are useful in the present invention.

As used herein, the term “amino acid” refers to a carboxylic acid bearing an amine functional groups. Amino acids include the diamino carboxylic acids described above. Amino acids include naturally occurring α-amino acids, wherein the amine is bound to the carbon adjacent to the carbonyl carbon of the carboxylic acid. Examples of naturally occurring α-amino acids include, but are not limited to, L-aspartic acid, L-glutamic acid, L-histidine, L-lysine, and L-arginine. Amino acids may also include the D-enantiomers of naturally occurring α-amino acids, as well as β-amino acids and other non-naturally occurring amino acids.

As used herein, the term “linker” refers to a chemical moiety that links one segment of a dendrimer conjugate to another. The types of bonds used to link the linker to the segments of the dendrimers include, but are not limited to, amides, amines, esters, carbamates, ureas, thioethers, thiocarbamates, thiocarbonate and thioureas. One of skill in the art will appreciate that other types of bonds are useful in the present invention.

As used herein, the term “oligomer” refers to five or fewer monomers, as described above, covalently linked together. The monomers may be linked together in a linear or branched fashion. The oligomer may function as a focal point for a branched segment of a telodendrimer.

As used herein, the term “hydrophobic group” refers to a chemical moiety that is water-insoluble or repelled by water. Examples of hydrophobic groups include, but are not limited to, long-chain alkanes and fatty acids, fluorocarbons, silicones, certain steroids such as cholesterol, and many polymers including, for example, polystyrene and polyisoprene.

As used herein, the term “hydrophilic group” refers to a chemical moiety that is water-soluble or attracted to water. Examples of hydrophilic groups include, but are not limited to, alcohols, short-chain carboxylic acids, quaternary amines, sulfonates, phosphates, sugars, and certain polymers such as PEG.

As used herein, the term “amphiphilic compound” refers to a compound having both hydrophobic portions and hydrophilic portions. For example, the amphiphilic compounds of the present invention can have one hydrophilic face of the compound and one hydrophobic face of the compound. Amphiphilic compounds useful in the present invention include, but are not limited to, cholic acid and cholic acid analogs and derivatives.

As used herein, the term “cholic acid” refers to (R)-4-((3R, 5S, 7R, 8R, 9S, 10S, 12S, 13R, 14S, 17R)-3,7,12-trihydroxy-10,13-dimethylhexadecahydro-1H-cyclopenta[a]phenanthren-17-yl)pentanoic acid. Cholic acid is also know as 3α,7α,12α-trihydroxy-5β-cholanoic acid; 3-α,7-α,12-α-Trihydroxy-5-β-cholan-24-oic acid; 17-β-(1-methyl-3-carboxypropyl)etiocholane-3 α,7 α,12 α-triol; cholalic acid; and cholalin. Cholic acid derivatives and analogs, such as allocholic acid, pythocholic acid, avicholic acid, deoxycholic acid, chenodeoxycholic acid, are also useful in the present invention. Cholic acid derivatives can be designed to modulate the properties of the nanocarriers resulting from telodendrimer assembly, such as micelle stability and membrane activity. For example, the cholic acid derivatives can have hydrophilic faces that are modified with one or more glycerol groups, aminopropanediol groups, or other groups.

As used herein, the terms “drug” or “therapeutic agent” refers to an agent capable of treating and/or ameliorating a condition or disease. The agent can be an inhibitor of vascularization. The agent may be a hydrophobic drug, which is any drug that repels water, such as a hydrophobic inhibitor of vascularization. The drugs of the present invention also include prodrug forms. One of skill in the art will appreciate that other drugs are useful in the present invention.

As used herein, the term “crosslinkable group” or “crosslinking group” refers to a functional group capable of binding to a similar or complementary group on another molecule, for example, a first crosslinkable group on a first dendritic polymer linking to a second crosslinkable group on a second dendritic polymer. Groups suitable as crosslinkable and crosslinking groups in the present invention include thiols such as cysteine, boronic acids and 1,2-diols including 1,2-dihydroxybenzenes such as catechol. When the crosslinkable and crosslinking groups combine, they form cross-linked bonds such as disulfides and boronic esters. Other crosslinkable and crosslinking groups are suitable in the present invention.

As used herein, the term “bond cleavage component” refers to an agent capable of cleaving the cross-linked bonds formed using the crosslinkable and crosslinking groups of the present invention. The bond cleavage component can be a reducing agent, such as glutathione, when the cross-linked bond is a disulfide, or mannitol when the cross-linked bond is formed from a boronic acid and 1,2-diol.

As used herein, the term “imaging agent” refers to chemicals that allow body organs, tissue or systems to be imaged. Exemplary imaging agents include paramagnetic agents, optical probes, and radionuclides.

As used herein, the terms “treat”, “treating” and “treatment” refers to any indicia of success in the treatment or amelioration of an injury, pathology, condition, or symptom (e.g., the size, growth, or presence of a vascular abnormality), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom or condition. The treatment or amelioration of symptoms can be based on any objective or subjective parameter; including, e.g., the result of a physical examination.

As used herein, the term “subject” refers to animals such as mammals, including, but not limited to, primates (e.g., humans), cows, sheep, goats, horses, dogs, cats, rabbits, rats, mice and the like. In certain embodiments, the subject is a human.

As used herein, the terms “therapeutically effective amount or dose” or “therapeutically sufficient amount or dose” or “effective or sufficient amount or dose” refer to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992), Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

As used herein, the term “photodynamic therapy” refers to use of nontoxic, light-sensitive compounds that become toxic to malignant or disease cells upon exposure to light. Photodynamic therapy involves a photosensitizer, a light source, and oxygen. Upon exposure to the light, the photosensitizer generates reactive oxygen species (singlet oxygen, an oxygen free radical) that react with and destroy the malignant tissue. A variety of photosensitizers can be used, including porphyrins or a derivative thereof, chlorophylls and dyes.

As used herein, the term “photothermal therapy” refers to use of nontoxic, light-sensitive compounds that generate heat upon exposure to light. Like photodynamic therapy, photothermal therapy involves a photosensitizer and a source of light, typically infrared. But photothermal therapy does not require oxygen. A variety of photosensitizers can be used, including porphyrins or a derivative thereof, chlorophylls and dyes.

As used herein, the term “local injection” refers to a method of administering in which an active agent (e.g., photosensitizer, nanocarrier, inhibitor of vascularization, etc., or a combination thereof) is injected at a site of treatment. For example, for treatment of a hemangioma, local injection can include injection of one or more active agent(s) into the hemangioma or proximal to (e.g., within 1, 2, 3, 5, 10, or 15 mm) the hemangioma.

As used herein, the term “effective amount of electromagnetic radiation” refers to an amount of electromagnetic radiation (i.e., visible, ultraviolet, or infrared) light that is effective to treat a vascular abnormality (e.g., hemangioma). The effective amount can be an amount effective to interact with a photosensitizer and cause heating, singlet oxygen generation, peroxide or hydroxyl radical generation, or direct energy or electron transfer from the photosensitizer to cellular and/or extracellular components and thereby induce treatment (e.g., cell death).

### DETAILED DESCRIPTION OF THE INVENTION

Infantile hemangiomas (IH) are the most common tumors of infancy affecting 10% of all children. They generally grow rapidly within the first few months of life in cosmetically sensitive areas causing distress to parents and placing children at a social developmental disadvantage. IH may cause visual and even life-threatening complications when present around the eyes and aerodigestive tract. Preventing rapid growth of IH early on can eliminate potential complications and dramatically improve quality of life for patients dramatically. Unfortunately, current treatment methods for IH face multiple challenges including poor overall efficacy and unwanted serious side effects.

This project defines a minimally invasive, high-efficacy treatment method for IHs and other vascular abnormalities. In one embodiment, a reproducible mouse model of IH is established and animals are administered a nanoporphyrin telodendrimer and treated with photodynamic therapy (PDT), photothermal therapy (PTT), or a combination of PDT and PIT. In another embodiment, mice exhibiting a model of IH are treated with a combination of PDT and pharmacotherapy delivered by a nanoparticle carrier, a combination of PTT and pharmacotherapy delivered by the nanoparticle carrier, or PDT and PTT in combination with pharmacotherapy delivered by the nanoparticle carrier (Table 1).

TABLE 1
Study groups (n = 16/group)

1. i.v saline (no laser, control)
2. i.v. saline + laser
3. i.v. NP + laser
4. i.v. propranolol-loaded NP (NP-Pro) + laser
5. i.v. NP + i.p. propranolol + laser
6. i.p. propranolol + laser

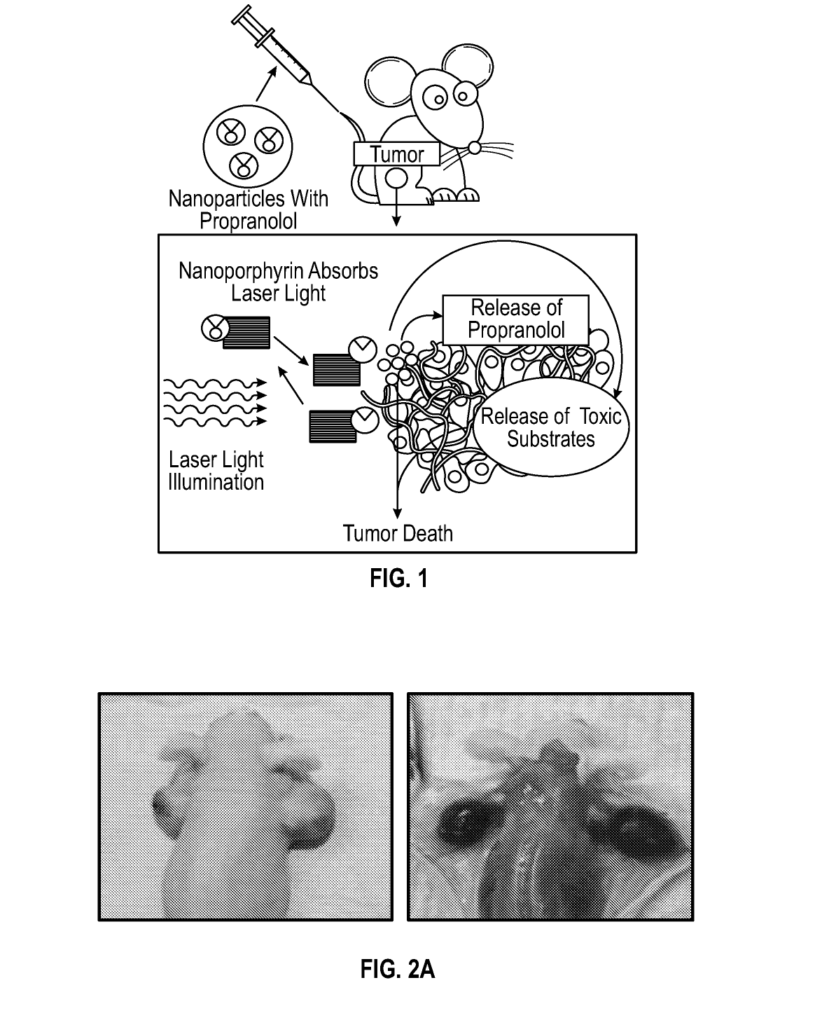

PDT destroys the target cells by triggering the formation of toxic reactive oxygen species (ROS) (FIG. 1). PTT acts in a similar way, but produces heat energy within the tumor. Propranolol is a beta blocking agent that can be used for the treatment of IHs. In one embodiment propranolol is loaded into nanoparticle carriers, which are injected into the blood circulation of the animals. The IHs are exposed to a near-infrared (NIR) laser. The NIR laser stimulates the nanoparticles to release propranolol at the lesion site and also initiate the synthesis of ROS and heat therefore producing a triple therapeutic effect.

Nanotechnology is an emerging area of research and holds great promise towards development of novel therapeutic agents and some nanoparticles are currently approved by FDA for cancer therapy. The technique described herein can provide control of IH and other vascular abnormalities via a novel multimodal treatment method.

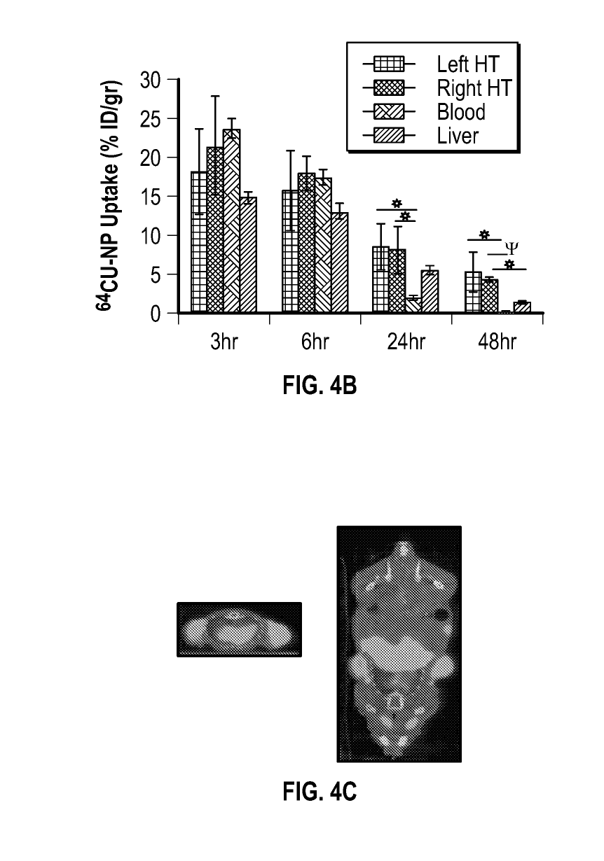

Without wishing to be bound by theory, it is believed that the IH vasculature is leaky and intravenously administered NP and/or nanoporphyrin telodendrimer can preferentially accumulate at the IH lesion (as shown by PET imaging, see FIG. 4), which can then be destroyed through PDT, PIT, or the combination thereof, without affecting, or substantially affecting, surrounding normal tissues. It is further believed that an inhibitor of vascularization, such as propranolol, encapsulated inside the NP (or co-administered simultaneously or sequentially with the NP and/or a nanoporphyrin telodendrimer via the same or a different route of administration) can further enhance the therapeutic efficacy of phototherapy (e.g., PDT, PIT, or the combination thereof).

Telodendrimers

The invention provides amphiphilic telodendrimer conjugates having a hydrophilic poly(ethylene glycol) (PEG) segment and a hydrophobic segment, and at least one porphyrin. The PEG segment can have a branched or linear architecture including one or more PEG chains. The hydrophobic segment of the telodendrimer can be provided by cholic acid, which has a hydrophobic face and a hydrophilic face. The porphyrin, cholic acid and the PEG are connected by oligomers and/or polymers that can contain a variety of acid repeats units. Typically, the oligomers and polymers comprise a diamino carboxylic acid, lysine. The telodendrimers can aggregate in solution to form micelles with a hydrophobic interior and a hydrophilic exterior. The micelles can be used as nanocarriers to deliver drugs or other agents having low water solubility. In some cases, the telodendrimer or nanocarrier is or contains a porphyrin modified telodendrimer as described in U.S. Patent Appl. No. 2014/0161719, the contents of which are hereby incorporated in the entirety for all purposes.

In some embodiments, the present invention provides conjugates having a polyethylene glycol (PEG) polymer; at least two amphiphilic compounds having both a hydrophilic face and a hydrophobic face; at least one porphyrin; optionally at least two crosslinking groups; and a dendritic polymer covalently attached to the PEG, the amphiphilic compounds, the porphyrin and the crosslinking groups, wherein each conjugate self-assembles in an aqueous solvent to form the nanocarrier such that a hydrophobic pocket is formed in the interior of the nanocarrier by the orientation of the hydrophobic face of each amphiphilic compound towards each other, wherein the PEG of each conjugate self-assembles on the exterior of the nanocarrier.

In some embodiments, the present invention provides a compound of formula I:

(B)ₖ-(PEG)ₘ-A(Y¹)ₚ-L¹-D-[Y²-L²-R]ₙ (I)

wherein B can be a binding ligand; each PEG can be a polyethyleneglycol (PEG) polymer having a molecular weight of 1-100 kDa; A includes at least one branched monomer unit X and can be linked to at least one PEG group; D can be a dendritic polymer having a single focal point group, a plurality of branched monomer units X and a plurality of end groups; each Y¹ and Y² can be absent or a crosslinkable group that can be boronic acid, dihydroxybenzene or a thiol; each L¹ and L² can independently be a bond or a linker, wherein L¹ can be linked to the focal point group of the dendritic polymer; each R can independently be the end group of the dendritic polymer, a porphyrin, a hydrophobic group, a hydrophilic group, an amphiphilic compound or a drug, wherein at least one R group can be a porphyrin; subscript k can be 0 or 1; subscript m can be an integer from 0 to 20; subscript n can be an integer from 2 to 20, wherein subscript n can be equal to the number of end groups on the dendritic polymer; and subscript p can be from 0 to 8. In some embodiments, subscript p is 0. A is absent, and L¹ is a bond or a linker linked to the focal point of the dendritic polymer.

Any suitable binding ligand can be used in the compounds of the present invention. For example, the binding ligand can target a particular organ, healthy tissue or disease tissue. Exemplary binding ligands include an anti-CD133 antibody, or the PLZ4 ligand, having the amino acid sequence QDGRMGF. See U.S. application Ser. No. 13/497,041, filed Sep. 23, 2010, now U.S. Publication No. 2012/0230994, the contents of which are hereby incorporated by reference in the entirety for all purposes.

The linkers L¹ and L² can include any suitable linker. In general, the linkers are bifunctional linkers, having two functional groups for reaction with each of two telodendrimer segments. In some embodiments, the linkers L¹ and L² can be a heterobifunctional linker. In some embodiments, the linkers L¹ and L² can be a homobifunctional linker. In some embodiments, the linkers L¹ and L¹ can independently be polyethylene glycol, polyserine, polyglycine, poly(serine-glycine), aliphatic amino acids, 6-amino hexanoic acid, 5-amino pentanoic acid, 4-amino butanoic acid or beta-alaninc. One of skill in the art will recognize that the size and chemical nature of the linker can be varied based on the structures of the telodendrimer segments to be linked.

In some embodiments, linkers L¹ and L² can have the formula:

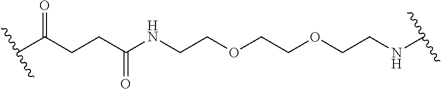 [SMILES: *NCCOCCOCCNC(=O)CCC(*)=O]

Polyethylene glycol (PEG) polymers of any size and architecture are useful in the nanocarriers of the present invention. In some embodiments, the PEG is from 1-100 kDa. In other embodiments, the PEG is from 1-10 kDa. In some other embodiments, the PEG is about 3 kDa. In still other embodiments, additional PEG polymers are linked to the amphiphilic compounds. For example, when the amphiphilic compound is cholic acid, up to 3 PEG polymers are linked to each cholic acid. The PEG polymers linked to the amphiphilic compounds are from 200-10,000 Da in size. In yet other embodiments, the PEG polymers linked to the amphiphilic compounds are from 1-5 kDa in size. One of skill in the art will appreciate that other PEG polymers and other hydrophilic polymers are useful in the present invention. PEG can be any suitable length.

The dendritic polymer can be any suitable dendritic polymer. The dendritic polymer can be made of branched monomer units including amino acids or other bifunctional AB2-type monomers, where A and B are two different functional groups capable of reacting together such that the resulting polymer chain has a branch point where an A-B bond is formed. In some embodiments, each branched monomer unit X can be a diamino carboxylic acid, a dihydroxy carboxylic acid and a hydroxyl amino carboxylic acid. In some embodiments, each diamino carboxylic acid can be 2,3-diamino propanoic acid, 2,4-diaminobutanoic acid, 2,5-diaminopentanoic acid (omithine), 2,6-diaminohexanoic acid (lysine), (2-Aminoethyl)-cysteine, 3-amino-2-aminomethyl propanoic acid, 3-amino-2-aminomethyl-2-methyl propanoic acid, 4-amino-2-(2-aminoethyl) butyric acid or 5-amino-2-(3-aminopropyl) pentanoic acid. In some embodiments, each dihydroxy carboxylic acid can be glyceric acid, 2,4-dihydroxybutyric acid, 2,2-Bis(hydroxymethyl)propionic acid, 2,2-Bis(hydroxymethyl)butyric acid, serine or threonine. In some embodiments, each hydroxyl amino carboxylic acid can be serine or homoserine. In some embodiments, the diamino carboxylic acid is an amino acid. In some embodiments, each branched monomer unit X is lysine.

The dendritic polymer of the telodendrimer can be any suitable generation of dendrimer, including generation 1, 2, 3, 4, 5, or more, where each “generation” of dendrimer refers to the number of branch points encountered between the focal point and the end group following one branch of the dendrimer. The dendritic polymer of the telodendrimer can also include partial-generations such as 1.5, 2.5, 3.5, 4.5, 5.5, etc., where a branch point of the dendrimer has only a single branch. The various architectures of the dendritic polymer can provide any suitable number of end groups, including, but not limited to, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or 32 end groups.

The focal point of a telodendrimer or a telodendrimer segment can be any suitable functional group. In some embodiments, the focal point includes a functional group that allows for attachment of the telodendrimer or telodendrimer segment to another segment. The focal point functional group can be a nucleophilic group including, but not limited to, an alcohol, an amine, a thiol, or a hydrazine. The focal point functional group may also be an electrophile such as an aldehyde, a carboxylic acid, or a carboxylic acid derivative including an acid chloride or an N-hydroxysuccinimidyl ester.

The R groups installed at the telodendrimer periphery can be any suitable chemical moiety, including porphyrins, hydrophilic groups, hydrophobic groups, or amphiphilic compounds, wherein at least one R group can be a porphyrin. Any suitable porphyrin can be used in the telodendrimers of the present invention. Representative porphyrins suitable in the present invention include, but are not limited to, pyropheophorbide-a, pheophorbide, chlorin e6, purpurin or purpurinimide. In some embodiments, the porphyrin can be pyrophcophorbide-a. Representative structures are shown below:

TABLE 2

| PORPH- | |
|---|---|
| YRIN | STRUCTURE |
| Porphyrin | 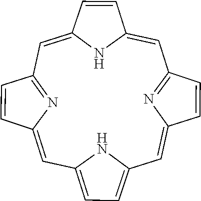 [SMILES: C1=Cc2cc3ccc(cc4nc(cc5ccc(cc1n2)[nH]5)C=C4)[nH]3] |
| Pyropheo- phorbide-a | 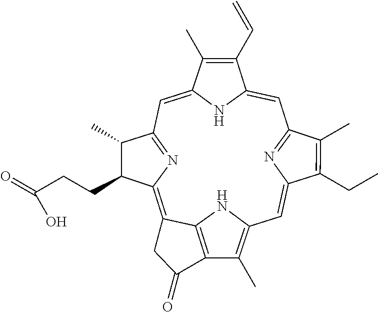 [SMILES: C=Cc1c(C)c2cc3nc(c4c5[nH]c(cc6nc(cc1[nH]2)C(C)=C6CC)c(C)c5C(=O)C4)[C@@H](CCC(=O)O)[C@@H]3C] |
| Pheo- phorbide | 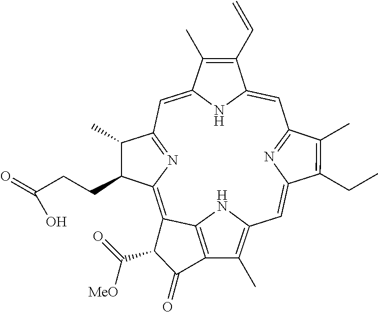 [SMILES: C=Cc1c(C)c2cc3nc(c4c5[nH]c(cc6nc(cc1[nH]2)C(C)=C6CC)c(C)c5C(=O)[C@@H]4C(=O)OC)[C@@H](CCC(=O)O)[C@@H]3C] |
| Chlorin e6 | 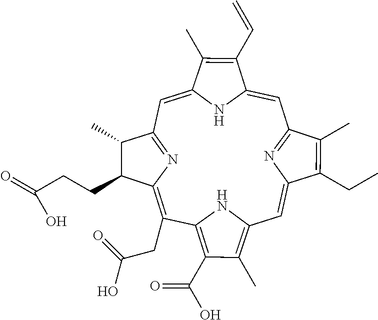 [SMILES: C=Cc1c(C)c2cc3nc(c(CC(=O)O)c4[nH]c(cc5nc(cc1[nH]2)C(C)=C5CC)c(C)c4C(=O)O)[C@@H](CCC(=O)O)[C@@H]3C] |
| Purpurin | 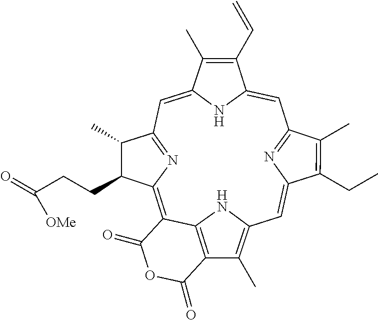 [SMILES: C=Cc1c(C)c2cc3nc(c4c5[nH]c(cc6nc(cc1[nH]2)C(C)=C6CC)c(C)c5C(=O)OC4=O)[C@@H](CCC(=O)OC)[C@@H]3C] |
| Purpuri- nimide | 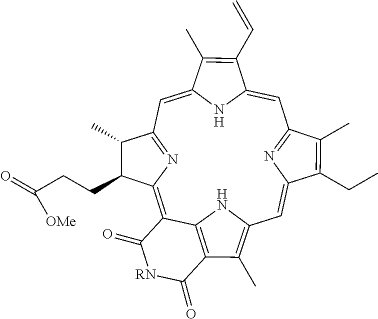 |

Examples of hydrophobic groups include, but are not limited to, long-chain alkanes and fatty acids, fluorocarbons, silicones, certain steroids such as cholesterol, and many polymers including, for example, polystyrene and polyisoprene. Examples of hydrophilic groups include, but are not limited to, alcohols, short-chain carboxylic acids, amines, sulfonates, phosphates, sugars, and certain polymers such as PEG. Examples of amphiphilic compounds include, but are not limited to, molecules that have one hydrophilic face and one hydrophobic face.

Amphiphilic compounds useful in the present invention include, but are not limited to, cholic acid and cholic acid analogs and derivatives. “Cholic acid” refers to (R)-4-((3R, 5S, 7R, 8R, 9S, 10S, 12S, 13R, 14S, 17R)-3,7,12-trihydroxy-10,13-dimethylhexadecahydro-1H-cyclopenta[a]phenanthren-17-yl)pentanoic acid, having the structure:

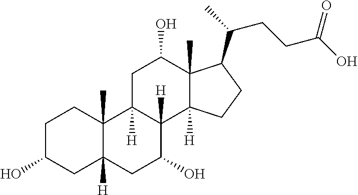

Cholic acid derivatives and analogs include, but are not limited to, allocholic acid, pythocholic acid, avicholic acid, deoxycholic acid, and chenodeoxycholic acid. Cholic acid derivatives can be designed to modulate the properties of the nanocarriers resulting from telodendrimer assembly, such as micelle stability and membrane activity. For example, the cholic acid derivatives can have hydrophilic faces that are modified with one or more glycerol groups, aminopropanediol groups, or other groups.

Telodendrimer end groups may also include drugs such as paclitaxel, doxorubicin, etoposide, irinotecan, SN-38, cyclosporin A, podophyllotoxin, carmustine, amphotericin, ixabepilone, patupilone (epothclone class), rapamycin, platinum drugs, vincristine, propranolol, an inhibitor of beta adrenergic receptor signaling, etc. One of skill in the art will appreciate that other drugs are useful in the present invention.

In some embodiments, each remaining R can be cholic acid, (3α, 5β, 7α, 12α)-7,12-dihydroxy-3-(2,3-dihydroxy-1-propoxy)-cholic acid, (3α, 5β, 7α, 12α)-7-hydroxy-3,12-di(2,3-dihydroxy-1-propoxy)-cholic acid, (3α, 5β, 7α, 12α)-7,12-dihydroxy-3-(3-amino-2-hydroxy-1-propoxy)-cholic acid, cholesterol formate, doxorubicin, or rhein. In other embodiments, each remaining R can be cholic acid.

The telodendrimer backbone can vary, depending on the number of branches and the number and chemical nature of the end groups and R groups, which will modulate solution conformation, rheological properties, and other characteristics. The telodendrimers can have any suitable number n of end groups and any suitable number of R groups. In some embodiments, n can be 2-70, or 2-50, or 2-30, or 2-10. In some embodiment, n is 2-20.

The telodendrimer can have a single type of R group on the periphery, or any combination of R groups in any suitable ratio. In general, at least half the number n of R groups are other than an end group. For example, at least half the number n of R groups can be a hydrophobic group, a hydrophilic group, an amphiphilic compound, a drug, or any combination thereof. In some embodiments, half the number n of R groups are amphiphilic compounds.

In some embodiments, the compound has the structure:

PEG-A-D-[Y²-L²-R]ₙ (Ia)

wherein each R can independently be a porphyrin, an amphiphilic compound or a drug, wherein at least one R group is a porphyrin.

In some embodiments, the compound has the structure:

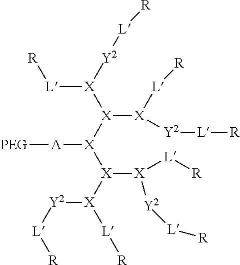

wherein PEG can be PEG5k, each branched monomer unit X can be lysine, A can be lysine, each L² can be a bond or linker Ebes, each Y¹ can be absent or can be cysteine; and each R can be a cholic acid or a porphyrin.

In some embodiments, the compound has the structure:

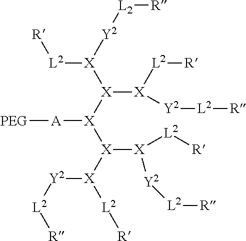

wherein each R′ can be cholic acid (CA), (3α, 5β, 7α, 12α)-7,12-dihydroxy-3-(2,3-dihydroxy-1-propoxy)-cholic acid (CA-4OH), (3α, 5β, 7α, 12α)-7-hydroxy-3,12-di(2,3-dihydroxy-1-propoxy)-cholic acid (CA-5OH) or (3α, 5β, 7α, 12α)-7,12-dihydroxy-3-(3-amino-2-hydroxy-1-propoxy)-cholic acid (CA-3OH—NH₂); and each R″ can be a porphyrin selected from the group consisting of pyropheophorbide-a, pheophorbide, chlorin e6, purpurin and purpurinimide. In other embodiments, the porphyrin can be pyropheophorbide-a. In some other embodiments, subscript k is 1. In some other embodiments, the compound can be:
(1) each L² is a bond, each Y² is absent, each R′ is cholic acid, each R″ is pyropheophorbide-a, and subscript k is 0;
(2) each L² is the linker Ebes, each Y² is absent, each R′ is cholic acid, each R″ is pyropheophorbide-a, and subscript k is 0;
(3) each L² is a bond, each Y² is cysteine, each R′ is cholic acid, each R″ is pyropheophorbide-a, and subscript k is 0,
(4) each L² is the linker Ebes, each Y² is cysteine, each R′ is cholic acid, each R″ is pyropheophorbide-a, and subscript k is 0;
(5) each L² is a bond, each Y² is absent, each R′ is cholic acid, each R″ is pyropheophorbide-a, and subscript k is 1;
(6) each L² is the linker Ebes, each Y² is absent, each R′ is cholic acid, each R″ is pyropheophorbide-a, and subscript k is 1;
(7) each L² is a bond, each Y¹ is cysteine, each R′ is cholic acid, each R″ is pyropheophorbide-a, and subscript k is 1; or
(8) each L² is the linker Ebes, each Y² is cysteine, each R′ is cholic acid, each R″ is pyropheophorbide-a, and subscript k is 1.

In some embodiments, the compound has the structure:

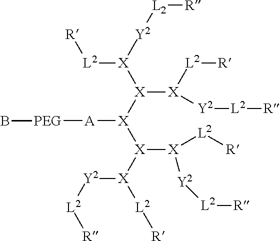

wherein each R′ can be cholic acid (CA), (3α, 5β, 7α, 12α)-7,12-dihydroxy-3-(2,3-dihydroxy-1-propoxy)-cholic acid (CA-4OH), (3α, 5β, 7α, 12α)-7-hydroxy-3,12-di(2,3-dihydroxy-1-propoxy)-cholic acid (CA-5OH) or (3α, 5β, 7α, 12α)-7,12-dihydroxy-3-(3-amino-2-hydroxy-1-propoxy)-cholic acid (CA-3OH—NH₂); and each R″ can be a porphyrin selected from the group consisting of pyropheophorbide-a, pheophorbide, chlorin e6, purpurin and purpurinimide. In other embodiments, the porphyrin can be pyropheophorbide-a. In some other embodiments, subscript k is 1. In some other embodiments, the compound can be:

TABLE 3

| | | PEG | | | | | | |
|---|---|---|---|---|---|---|---|---|
| Compound | B | (mw) | A | X | L² | Y² | R′ | R″ |
| 1 | absent | 5k | lysine | lysine | bond | absent | cholic acid | pyropheophorbide-a |
| 2 | absent | 5k | lysine | lysine | Ebes | absent | cholic acid | pyropheophorbide-a |
| 3 | absent | 5k | lysine | lysine | bond | cysteine | cholic acid | pyropheophorbide-a |
| 4 | absent | 5k | lysine | lysine | Ebes | cysteine | cholic acid | pyropheophorbide-a |
| 5 | PLZ4 | 5k | lysine | lysine | bond | absent | cholic acid | pyropheophorbide-a |
| 6 | PLZ4 | 5k | lysine | lysine | Ebes | absent | cholic acid | pyropheophorbide-a |
| 7 | PLZ4 | 5k | lysine | lysine | bond | cysteine | cholic acid | pyropheophorbide-a |
| 8 | PLZ4 | 5k | lysine | lysine | Ebes | cysteine | cholic acid | pyropheophorbide-a |
| 9 | absent | 5k | absent | lysine | bond | absent | cholic acid | pyropheophorbide-a |
| 10 | absent | 5k | absent | lysine | Ebes | absent | cholic acid | pyropheophorbide-a |
| 11 | absent | 5k | absent | lysine | bond | cysteine | cholic acid | pyropheophorbide-a |
| 12 | absent | 5k | absent | lysine | Ebes | cysteine | cholic acid | pyropheophorbide-a |
| 13 | PLZ4 | 5k | absent | lysine | bond | absent | cholic acid | pyropheophorbide-a |
| 14 | PLZ4 | 5k | absent | lysine | Ebes | absent | cholic acid | pyropheophorbide-a |
| 15 | PLZ4 | 5k | absent | lysine | bond | cysteine | cholic acid | pyropheophorbide-a |
| 16 | PLZ4 | 5k | absent | lysine | Ebes | cysteine | cholic acid | pyropheophorbide-a |

In some embodiments of the foregoing porphyrin telodendrimers, as illustrated in rows 9-16 of Table 3, A is absent. In such embodiments, the compound can have the following structure:

PEG-D-[Y²-L²-R]ₙ (Ib)

wherein each R can independently be a porphyrin, an amphiphilic compound or a drug, wherein at least one R group is a porphyrin.

In some embodiments, the compound has the structure:

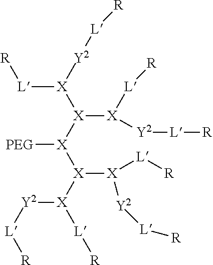

wherein PEG can be PEG5k, each branched monomer unit X can be lysine, each L² can be a bond or linker Ebes, each Y² can be absent or can be cysteine; and each R can be a cholic acid or a porphyrin.

In some embodiments, the compound has the structure:

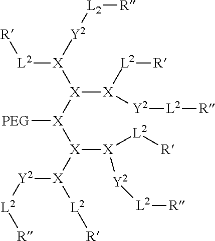

wherein each R′ can be cholic acid (CA), (3α, 5β, 7α, 12α)-7,12-dihydroxy-3-(2,3-dihydroxy-1-propoxy)-cholic acid (CA-4OH), (3α, 5β, 7α, 12α)-7-hydroxy-3,12-di(2,3-dihydroxy-1-propoxy)-cholic acid (CA-5OH) or (3α, 5β, 7α, 12α)-7,12-dihydroxy-3-(3-amino-2-hydroxy-1-propoxy)-cholic acid (CA-3OH—NH₂); and each R″ can be a porphyrin selected from the group consisting of pyropheophorbide-a, pheophorbide, chlorin e6, purpurin and purpurinimide. In other embodiments, the porphyrin can be pyropheophorbide-a. In some other embodiments, subscript k is 1. In some other embodiments, the compound can be:
(9) each L² is a bond, each Y² is absent, each R′ is cholic acid, each R″ is pyropheophorbide-a, and subscript k is 0;
(10) each L² is the linker Ebes, each Y² is absent, each R′ is cholic acid, each R″ is pyropheophorbide-a, and subscript k is 0;
(11) each L² is a bond, each Y² is cysteine, each R′ is cholic acid, each R″ is pyropheophorbide-a, and subscript k is 0.
(12) each L² is the linker Ebes, each Y² is cysteine, each R′ is cholic acid, each R″ is pyropheophorbide-a, and subscript k is 0;
(13) each L² is a bond, each Y² is absent, each R′ is cholic acid, each R″ is pyropheophorbide-a, and subscript k is 1;
(14) each L² is the linker Ebes, each Y² is absent, each R′ is cholic acid, each R″ is pyropheophorbide-a, and subscript k is 1;
(15) each L² is a bond, each Y² is cysteine, each R′ is cholic acid, each R″ is pyropheophorbide-a, and subscript k is 1; or
(16) each L² is the linker Ebes, each Y² is cysteine, each R′ is cholic acid, each R″ is pyropheophorbide-a, and subscript k is 1.

In some embodiments, the compound has the structure:

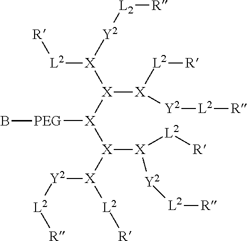

wherein each R′ can be cholic acid (CA), (3α, 5β, 7α, 12α)-7,12-dihydroxy-3-(2,3-dihydroxy-1-propoxy)-cholic acid (CA-4OH), (3α, 5β, 7α, 12α)-7-hydroxy-3,12-di(2,3-dihydroxy-1-propoxy)-cholic acid (CA-5OH) or (3α, 5β, 7α, 12α)-7,12-dihydroxy-3-(3-amino-2-hydroxy-1-propoxy)-cholic acid (CA-3OH—NH₂); and each R″ can be a porphyrin selected from the group consisting of pyropheophorbide-a, pheophorbide, chlorin e6, purpurin and purpurinimide. In other embodiments, the porphyrin can be pyropheophorbide-a. In some other embodiments, subscript k is 1. With reference to Formula I, Formula Ib can be considered a subset in which subscript p is 0, and A is absent or the focal point monomer X of D.

The compounds of the present invention can also include a metal cation chelated to the porphyrin. Any suitable metal can be chelated by the porphyrin. Metals useful in the present invention include the alkali metals, alkali earth metals, transition metals and post-transition metals. Alkali metals include Li, Na, K, Rb and Cs. Alkaline earth metals include Be, Mg, Ca, Sr and Ba. Transition metals include Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Hg and Ac. Post-transition metals include Al, Ga, In, Tl, Ge, Sn, Pb, Sb, Bi, and Po. Radionuclides of any of these metals can also be chelated by the porphyrins. In some embodiments, the a metal cation can be chelated to the porphyrin. In other embodiments, the metal cation can be a radio-metal cation. In some other embodiments, the radio-metal cation chelated to the porphyrin can be ⁶⁴Cu, ⁶⁷Cu, ¹⁷⁷Lu, ⁶⁷Ga, ¹¹¹In, and ⁹⁰Yt.

Telodendrimers with Branched PEG Moieties

The telodendrimers of the present invention contain two branched segments that are linked together at their focal points. Generally, the telodendrimers include any telodendrimer as described above or as described previously (WO 2010/039496) and branched PEG segment containing two or more PEG chains bound to an oligomer focal point.

The dendritic polymer of the telodendrimer can be any suitable generation of dendrimer, including generation 1, 2, 3, 4, 5, or more, where each “generation” of dendrimer refers to the number of branch points encountered between the focal point and the end group following one branch of the dendrimer. The dendritic polymer of the telodendrimer can also include partial-generations such as 1.5, 2.5, 3.5, 4.5, 5.5, etc., where a branch point of the dendrimer has only a single branch. The various architectures of the dendritic polymer can provide any suitable number of end groups, including, but not limited to, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or 32 end groups.

In some embodiments, the compound can be:

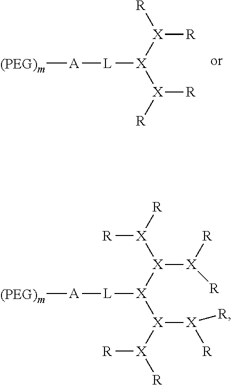

wherein each branched monomer unit X is lysine.

In some embodiments, the compound can be:

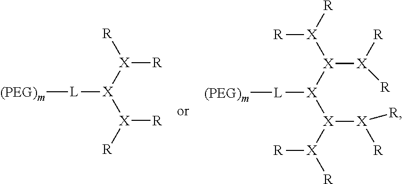

wherein each branched monomer unit X is lysine. In some embodiments, each R is independently cholic acid or a porphyrin.

In some embodiments, the compound can be:

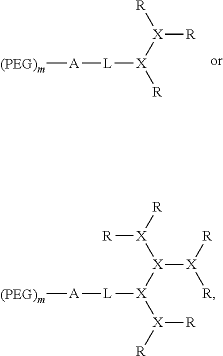

wherein each branched monomer unit X is lysine.

The PEG-oligomer unit in the telodendrimers may contain any suitable number of PEG moieties. PEG moieties may be installed site-selectively at various positions on the oligomer using orthogonal protecting groups. In some embodiments, the (PEG)ₘ-A portion of the compound can be:

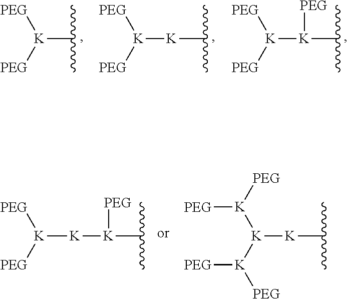

wherein each K is lysine.

In some embodiments, the telodendrimer can be:

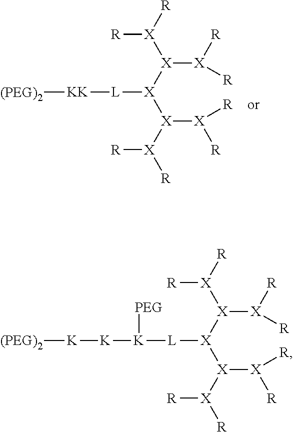

wherein each K is lysine; each PEG is PEG2k; each branched monomer unit X is lysine; each R is cholic acid; and linker L has the formula:

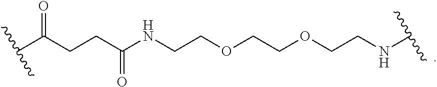

Nanocarriers

The telodendrimers of the present invention aggregate to form nanocarriers with a hydrophobic core and a hydrophilic exterior. In some embodiments, the invention provides a nanocarrier having an interior and an exterior, the nanocarrier comprising a plurality of the dendrimer conjugates of the invention, wherein each compound self-assembles in an aqueous solvent to form the nanocarrier such that a hydrophobic pocket is formed in the interior of the nanocarrier, and wherein the PEG of each compound self-assembles on the exterior of the nanocarrier.

In some embodiments, each conjugate of the nanocarrier have a polyethylene glycol (PEG) polymer; at least two amphiphilic compounds having both a hydrophilic face and a hydrophobic face; at least one porphyrin; optionally at least two crosslinking groups; and a dendritic polymer covalently attached to the PEG, the amphiphilic compounds, the porphyrin and the crosslinking groups, wherein each conjugate self-assembles in an aqueous solvent to form the nanocarrier such that a hydrophobic pocket is formed in the interior of the nanocarrier by the orientation of the hydrophobic face of each amphiphilic compound towards each other, wherein the PEG of each conjugate self-assembles on the exterior of the nanocarrier. In other embodiments, each conjugate is a compound of formula I.

In some embodiments, the nanocarrier includes a hydrophobic drug or an imaging agent, such that the hydrophobic drug or imaging agent is sequestered in the hydrophobic pocket of the nanocarrier. Hydrophobic drugs useful in the nanocarrier of the present invention includes any drug having low water solubility. In some embodiments, the drug in the nanocarrier can be vincristine. In some embodiments, the drug in the nanocarrier can be an inhibitor of vascularization. The inhibitor of vascularization can be selected from the group consisting of propranolol, metoprolol, atenolol, acebutolol, nadolol, pindolol, alprenolol, timolol, an inhibitor of extracellular-signal-regulated kinase (ERK) signaling (e.g., an inhibitor of signaling of ERK 1, 2, or 3, or a combination thereof), or an inhibitor of vascular endothelial growth factor receptor type-2 (VEGFR-2). In some embodiments, the drug in the nanocarrier can be an inhibitor of beta adrenergic receptor signaling.

In some embodiments, the nanocarrier includes at least one monomer unit that is optionally linked to an optical probe, a radionuclide, a paramagnetic agent, a metal chelate or a drug. The drug can be a variety of hydrophilic or hydrophobic drugs, and is not limited to the hydrophobic drugs that are sequestered in the interior of the nanocarriers of the present invention.

Drugs that can be sequestered in the nanocarriers or linked to the conjugates of the present invention include, but are not limited to, cytostatic agents, cytotoxic agents (such as for example, but not limited to, DNA interactive agents (such as cisplatin or doxorubicin)); taxanes (e.g. taxotere, taxol); topoisomerase II inhibitors (such as etoposide); topoisomerase I inhibitors (such as irinotecan (or CPT-11), camptostar, or topotecan); tubulin interacting agents (such as paclitaxel, docetaxel or the epothilones); hormonal agents (such as tamoxifen); thymidilate synthase inhibitors (such as 5-fluorouracil); anti-metabolites (such as methotrexate); alkylating agents (such as temozolomide (TEMODAR™ from Schering-Plough Corporation, Kenilworth, N.J.), cyclophosphamide); aromatase combinations; ara-C, adriamycin, cytoxan, and gemcitabine. Other drugs useful in the nanocarrier of the present invention include but are not limited to Uracil mustard, Chlormethine, Ifosfamide, Melphalan, Chlorambucil, Pipobroman, Triethylenemelamine, Triethylenethiophosphoramine, Busulfan, Carmustine. Lomustine, Streptozocin, Dacarbazine, Floxuridine, Cytarabine, 6-Mercaptopurine, 6-Thioguanine, Fludarabine phosphate, oxaliplatin, leucovirin, oxaliplatin (ELOXATIN™ from Sanofi-Synthelabo Pharmaceuticals, France), Pentostatine. Vinblastine, Vincristine, Vindesine, Bleomycin, Dactinomycin, Daunorubicin, Doxorubicin, Epirubicin, Idarubicin. Mithramycin, Deoxycofonnycin, Mitomycin-C, L-Asparaginase, Teniposide 17.alpha.-Ethinylcstradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserclin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, or Hexamethylmelamine. Other drugs that can be sequestered into or conjugated to a nanocarrier of the present invention include, but are not limited to, VEGFR-2 inhibitors such as sorafenib, sunitinib, apatinib, lenvatinib, motesanib, pazopanib, regorafenib, and the like. Other drugs that can be sequestered into or conjugated to a nanocarrier of the present invention include, but are not limited to, inhibitors of ERK signaling such as an inhibitor of ERK1/2, an inhibitor of BRAF, an inhibitor of MEK, PLX4720, PLX4032 (vemurafenib), AZD6244, GSK2118436 and U0126, and the like. BRAF inhibitors include, but are not limited to dasatinib, erlotinib, geftinib, imatinib, lapatinib, sorafenib, sunitinib, dexanabinol, PD-325901, XL518, PD-318088, RG7204, GDC-0879, and sorafenib losylate (Bay 43-9006) or a derivative or pharmaceutically acceptable salt thereof. These and other inhibitors of BRAF, as well as non-limiting examples of their methods of manufacture, are described in U.S. Patent Publication Nos. US 2005/0176740, US 2011/0020217, US 2007/0078121, US 2011/0118298. U.S. Pat. No. 4,876,276; and International Patent Applications WO 02/24680, WO 03/022840, WO 07/002,325 the contents of which are herein incorporated by reference in the entirety for all purposes. Prodrug forms are also useful in the present invention.

Other drugs useful in the present invention also include radionuclides, such as ⁶⁷Cu, ⁹⁰Y, ¹²³I, ¹²⁵I, ¹³¹I, ¹⁷⁷Lu, ¹⁸⁸Re, ¹⁸⁶Re and ²¹¹At. In some embodiments, a radionuclide can act therapeutically as a drug and as an imaging agent.

Imaging agents include paramagnetic agents, optical probes and radionuclides. Paramagnetic agents include iron particles, such as iron nanoparticles that are sequestered in the hydrophobic pocket of the nanocarrier.

In some embodiments, the conjugates can be crosslinked via the crosslinking groups. The crosslinking groups can be any suitable crosslinking group, as described above. In some embodiments, the crosslinking groups can be thiol, boronic acid or dihydroxybenzene. In some embodiments, the crosslinking groups can be thiol. In some embodiments, a first set of conjugates includes boronic acid crosslinking groups, and a second set of conjugates includes dihydroxybenzene crosslinking groups. In some embodiments, each conjugate of the nanocarrier includes at least two cholic acids, at least two pryopheophorbide-a groups, and at least two crosslinking groups, wherein the conjugates of the nanocarrier are crosslinked via the crosslinking groups.

The nanocarriers can include any suitable porphyrin, as described above. In some embodiments, the porphyrin can be pyrpheophorbide-a. In some embodiments, the porphyrin groups can be chelated to a metal, as described above. Any suitable metal can be chelated to the porphyrins, including radioactive and non-radioactive metals, as described above. In some embodiments, the nanocarriers include a metal chelated to at least one of the pyropheophorbide-a groups.

Some embodiments of the invention provide nanocarriers wherein each amphiphilic compound R is independently cholic acid, allocholic acid, pythocholic acid, avicholic acid, deoxycholic acid, or chenodeoxycholic acid.

The nanocarriers of the present invention can also include a binding ligand for binding to a target moiety. The binding ligand can be linked to one of the conjugates of the nanocarrier, or can be separate. Any suitable binding ligand can be used in the compounds of the present invention, as described above. For example, the binding ligand can target a particular organ, healthy tissue or disease tissue. Exemplary binding ligands include an anti-CD133 antibody, or the PLZ4 ligand, having the amino acid sequence cQDGRMGFc. In some embodiments, the nanocarrier including at least one binding conjugate including a polyethylene glycol (PEG) polymer, a binding ligand linked to the PEG polymer, at least two amphiphilic compounds having both a hydrophilic face and a hydrophobic face, a dendritic polymer covalently attached to the PEG and the amphiphilic compounds, wherein each binding conjugate self-assembles with the first conjugates in an aqueous solvent to form the nanocarrier such that a hydrophobic pocket is formed in the interior of the nanocarrier by the orientation of the hydrophobic face of each amphiphilic compound towards each other, wherein the PEG of each conjugate self-assembles on the exterior of the nanocarrier.

Formulations

The nanocarriers, telodendrimers, inhibitors of vascularization, or combinations thereof, of the present invention can be formulated in a variety of different manners known to one of skill in the art. Pharmaceutically acceptable carriers are determined in part by the particular composition being administered, as well as by the particular method used to administer the composition. Accordingly, there are a wide variety of suitable formulations of pharmaceutical compositions of the present invention (see. e.g., Remington's Pharmaceutical Sciences, 20^{th} ed., 2003, supra). Effective formulations include oral and nasal formulations, formulations for parenteral administration, and compositions formulated for with extended release.

Formulations suitable for oral administration can consist of (a) liquid solutions, such as an effective amount of a compound of the present invention suspended in diluents, such as water, saline or PEG 400; (b) capsules, sachets, depots or tablets, each containing a predetermined amount of the active ingredient, as liquids, solids, granules or gelatin; (c) suspensions in an appropriate liquid; (d) suitable emulsions; and (e) patches. The liquid solutions described above can be sterile solutions. The pharmaceutical forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents. Preferred pharmaceutical preparations can deliver the compounds of the invention in a sustained release formulation.

Pharmaceutical preparations useful in the present invention also include extended-release formulations. In some embodiments, extended-release formulations useful in the present invention are described in U.S. Pat. No. 6,699,508, which can be prepared according to U.S. Pat. No. 7,125,567, both patents incorporated herein by reference.

The pharmaceutical preparations are typically delivered to a mammal, including humans and non-human mammals. Non-human mammals treated using the present methods include domesticated animals (i.e., canine, feline, murine, rodentia, and lagomorpha) and agricultural animals (bovine, equine, ovine, porcine).

In practicing the methods of the present invention, the pharmaceutical compositions can be used alone, or in combination with other therapeutic or diagnostic agents.

Methods of Treating

The nanocarriers of the present invention can be used to treat any disease requiring the administration of a drug, such as by sequestering a hydrophobic drug in the interior of the nanocarrier, or by covalent attachment of a drug to a conjugate of the nanocarrier. The nanocarriers can also be used for imaging, by sequestering an imaging agent in the interior of the nanocarrier, or by attaching the imaging agent to a conjugate of the nanocarrier.

In some embodiments, the present invention provides a method of treating a disease, including administering to a subject in need of such treatment, a therapeutically effective amount of a nanocarrier and/or telodendrimers of the present invention. The nanocarrier and/or telodendrimer can include a drug. The drug can be a covalently attached to a conjugate of the nanocarrier and/or telodendrimers. In some embodiments, the drug is a hydrophobic drug sequestered in the interior of the nanocarrier. In some embodiments, the nanocarrier also includes an imaging agent. The imaging agent can be a covalently attached to a conjugate of the nanocarrier, or the imaging agent can be sequestered in the interior of the nanocarrier. In some other embodiments, both a hydrophobic drug and an imaging agent are sequestered in the interior of the nanocarrier. In still other embodiments, both a drug and an imaging agent are covalently linked to a conjugate or conjugates of the nanocarrier. In yet other embodiments, the nanocarrier can also include a radionuclide.

In some embodiments, the disease is treated by administering a telodendrimer (e.g., porphyrin containing telodendrimer) or a nanocarrier of one or more teledendrimers (e.g., and optionally one or more drugs, imaging agents, or combinations thereof) to the patient and performing photodynamic or photothermal therapy. In some embodiments, the disease is treated by administering a telodendrimer and an inhibitor of vascularization to the patient and performing photodynamic or photothermal therapy. In some cases, the telodendrimer and inhibitor of vascularization are administered at the same time and via the same route. For example, the inhibitor of vascularization can be sequestered in the interior of a nanocarrier comprising the telodendrimer, which is administered to the subject. As another example, a telodendrimer or nanocarrier comprising telodendrimer and an inhibitor of vascularization that is not sequestered can be administered as a mixture. As yet another example, a telodendrimer or nanocarrier comprising telodendrimer and an inhibitor of vascularization that is not sequestered can be administered sequentially via the same route. In some cases, the telodendrimer and nanocarrier are simultaneously or sequentially administered via different routes.

Telodendrimers, nanocarriers, drugs, imaging agents, and combinations thereof can be administered by local injection (e.g., intradermal, subcutaneous, intra-tumoral, or intra-hemangiomal), topical administration, systemically (e.g., intravenous). In some cases, a telodendrimer and/or nanocarrier is administered by local injection or topical administration, and a drug (e.g., inhibitor of vascularization) is administered systemically. In some cases, a telodendrimer and/or nanocarrier is administered by local injection and a drug (e.g., timolol) is administered topically.

The methods of treating using the nanocarriers and/or telodendrimers of the present invention also includes treating a disease or conditions, such as a vascular abnormality (e.g., vascular tumor or hemangioma), by photodynamic therapy or photothermal therapy. The methods generally involve administering a nanocarrier and/or telodendrimers of the present invention to a subject, and then exposing the subject to radiation having a specific wavelength to induce the photodynamic or photothermal therapy. Upon exposure to the radiation or light, porphyrins or other light absorbing moieties present in the nanocarriers and/or telodendrimers of the present invention, either complexed to a metal or not, generate reactive singlet oxygen, hydroxyl radicals, or peroxides suitable for photodynamic therapy, generate heat sufficient for photothermal therapy, or otherwise cause direct energy or electron transfer from the photosensitizer to cellular and/or extracellular components sufficient for photodynamic and/or photothermal therapy.

In some embodiments, the present invention provides a method of treating a disease via photodynamic or photothermal therapy, including administering to a subject in need thereof, a therapeutically effective amount of a nanocarrier and/or telodendrimers of the present invention, and optionally a drug (e.g., inhibitor of vascularization), and exposing the subject to electromagnetic radiation, thereby treating the disease via photodynamic or photothermal therapy. In some embodiments, the method is a method of treating a disease via photodynamic therapy. In other embodiments, the method is a method of treating a disease via photothermal therapy. In some cases, the electromagnetic radiation has a controlled wavelength. In some cases, the vascular abnormality is exposed to electromagnetic radiation from a laser, such as a diode laser (e.g., a 405 nm diode laser). In some cases, the vascular abnormality is exposed to electromagnetic radiation from a light emitting diode (e.g., a 410 nm light emitting diode). In some cases, the electromagnetic radiation has or contains photons having a wavelength of about 405 nm (e.g., between about 400 and about 420 nm) or about 680 nm (e.g., between about 600 and about 700), or a combination thereof.

In other embodiments, the present invention provides a method of treating a disease via sonodynamic therapy, including administering to a subject in need thereof, a therapeutically effective amount of a nanocarrier and/or telodendrimers of the present invention, and exposing the subject to a sonic wave, thereby treating the disease via sonodynamic therapy.

Any suitable conjugate or nanocarrier can be used in the methods of the present invention. In some embodiments, each conjugate of the nanocarrier includes at least one porphyrin group. In some embodiments, each conjugate of the nanocarrier includes at least two cholic acids, at least two pryopheophorbide-a groups, at least two crosslinking groups, and a metal chelated to at least one of the pyropheophorbide-a groups, wherein the conjugates of the nanocarrier are crosslinked via the crosslinking groups.

Methods of Administration

The nanocarriers, telodendrimers, drugs (e.g., inhibitors of vascularization), or combinations thereof, of the present invention can be administered as frequently as necessary, including hourly, daily, weekly or monthly. The compounds utilized in the pharmaceutical method of the invention are administered at the initial dosage of about 0.0001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound being employed. For example, dosages can be empirically determined considering the type and stage of disease diagnosed in a particular patient. The dose administered to a patient, in the context of the present invention should be sufficient to effect a beneficial therapeutic response in the patient over time. The size of the dose also will be determined by the existence, nature, and extent of any adverse side-effects that accompany the administration of a particular compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired. Doses can be given daily, or on alternate days, as determined by the treating physician. Doses can also be given on a regular or continuous basis over longer periods of time (weeks, months or years), such as through the use of a subdermal capsule, sachet or depot, or via a patch or pump.

The pharmaceutical compositions can be administered to the patient in a variety of ways, including topically, parenterally, systemically, intravenously, intradermally, subcutaneously, intramuscularly, colonically, rectally or intraperitoneally. Preferably, the pharmaceutical compositions are administered parenterally, topically, intravenously, intramuscularly, subcutaneously, orally, or nasally, such as via inhalation. In some cases, a nanoporphyrin telodendrimer, or a nanocarrier containing the nanoporphyrin telodendrimer is administered via one route (e.g., topically, parenterally, systemically, intravenously, intradermally, subcutaneously, intramuscularly, colonically, rectally or intraperitoneally), and a drug (e.g., inhibitor of vascularization) is administered via another route. In some cases, a nanocarrier comprised of nanoporphyrin telodendrimers and having a drug (e.g., an inhibitor of vascularization) sequestered therein is administered via one route, and a second drug (e.g., a second inhibitor of vascularization) is administered via another route.

In practicing the methods of the present invention, the pharmaceutical compositions can be used alone, or in combination with other therapeutic or diagnostic agents. The additional drugs used in the combination protocols of the present invention can be administered separately or one or more of the drugs used in the combination protocols can be administered together, such as in an admixture. Where one or more drugs are administered separately, the timing and schedule of administration of each drug can vary. The other therapeutic or diagnostic agents can be administered at the same time as the compounds of the present invention, separately or at different times.

Methods of Imaging

In some embodiments, the present invention provides a method of imaging, including administering to a subject to be imaged, an effective amount of a nanocarrier and/or telodendrimer of the present invention, wherein the nanocarrier and/or telodendrimer includes an imaging agent. In other embodiments, the method of treating and the method of imaging are accomplished simultaneously using a nanocarrier and/or telodendrimer having both a drug and an imaging agent.

Exemplary imaging agents include paramagnetic agents, optical probes, and radionuclides. Paramagnetic agents imaging agents that are magnetic under an externally applied field. Examples of paramagnetic agents include, but are not limited to, iron particles including nanoparticles. Optical probes are fluorescent compounds that can be detected by excitation at one wavelength of radiation and detection at a second, different, wavelength of radiation. Optical probes useful in the present invention include, but are not limited to, Cy5.5, Alexa 680, Cy5, DiD (1,1′-dioctadecyl-3,3,3′,3′-tetramethylindodicarbocyanine perchlorate) and DiR (1,1′-dioctadecyl-3,3,3′,3′-tetramethylindotricarbocyanine iodide). Other optical probes include quantum dots. Radionuclides are elements that undergo radioactive decay. Radionuclides useful in the present invention include, but are not limited to, ³H, ¹¹C, ¹³N, ¹⁸F, ¹⁹F, ⁶⁰Co, ⁶⁴Cu, ⁶⁷Cu, ⁶⁸Ga, ⁸²Rb, ⁹⁰Sr, ⁹⁰Y, ⁹⁹Tc, ^{99m}Tc, ¹¹¹In, ¹²³I, ¹²⁴I, ¹²⁵I, ¹²⁹I, ¹³¹I, ¹³⁷Cs, ¹⁷⁷Lu, ¹⁸⁶Re, ¹⁸⁸Re, ²¹¹At, Rn, Ra, Th, U, Pu and ²⁴¹Am.

### EXAMPLES

### Example 1: Treatment and Imaging of Vascular Abnormalities with Porphyrin Modified Telodendrimer

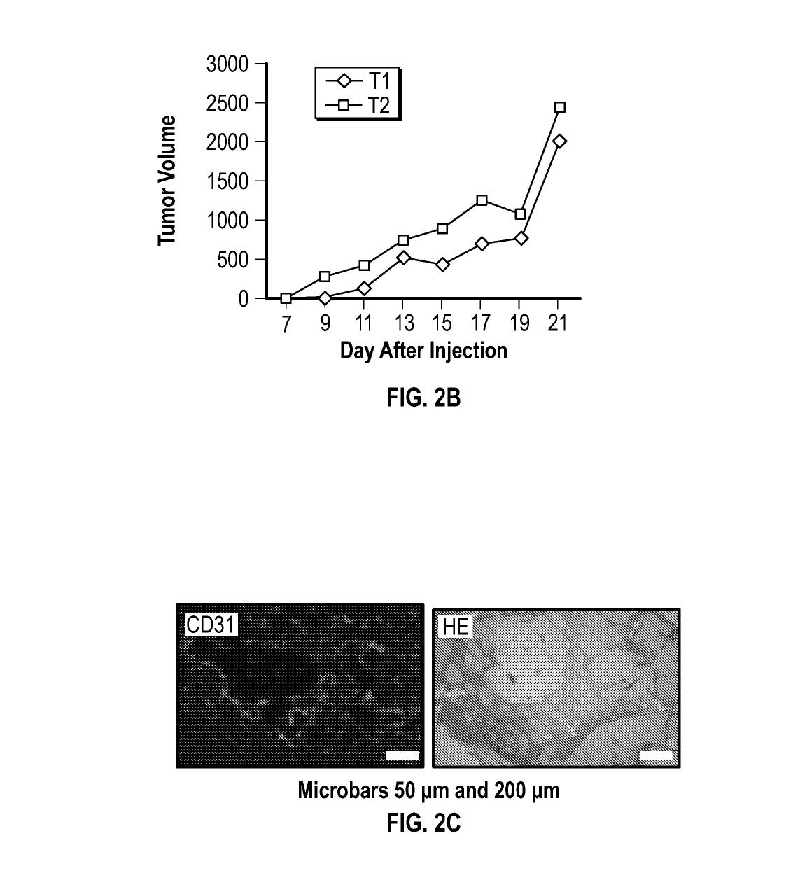

A reproducible animal model of infantile hemangioma (IH) has been established using a mouse hemangioendothelioma cell line (EOMA cells, American Type Culture Collection [ATCC®, CRL-2587™], Manassas, Va.). A reliable tumor growth could be induced by intradermal (i.d.) injection of 1.5×10⁶ EOMA cells to bilateral dorsal axillary regions of nude mice (FIG. 2A). With this method, tumor growth commenced 1 week subsequent to injection, and tumors continued to grow until the day 21, when animals were humanely euthanized due to deterioration in their systemic health resultant from increased tumor size (FIG. 2B). Histological staining for CD31 (top) and hematoxylin and eosin (HE, bottom) demonstrated the highly vascular characteristic structure of IH (FIG. 2C). Therefore, the model mirrors characteristics of natural IH, making it an excellent platform to test the teleodendrimer therapy.

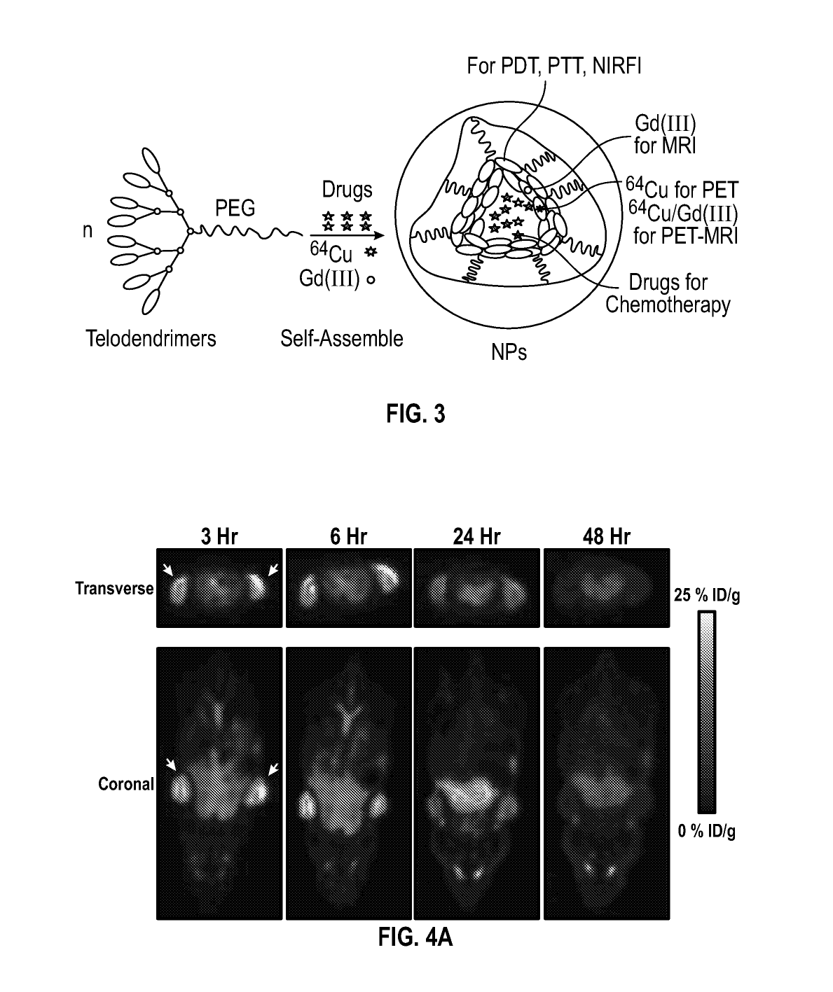

Novel multifunctional porphyrin-based micellar nanoparticle, nanoporphyrin (NP) compounds have recently been reported (FIG. 3). Porphyrin compounds are naturally found in the human body (e.g. hemoglobin). NP has two absorption peaks, one at 405 nm and one in the near-infrared (NIR) range with peak at ˜680 nm and also generates reactive oxygen species (ROS) and heat in phosphate buffered saline (PBS) when irradiated with a laser. The inventors have shown that NP-mediated PDT therapy led to significant tumor inhibition by using a much lower dose of light and photosensitizer compared with recently reported porphyrin formulations, e.g. liposomal porphyrins. Furthermore, NP-mediated combination chemotherapy and PDT was dramatically more efficacious than single treatment alone. This novel PDT agent is far superior than existing FDA approved photosensitizers and more effective in IH due to IH's superficial location and availability for illumination with laser.

All NP-based experiments described in this Example were performed using a NP telodendrimer of the formula PEG^{5k}-Por₄-CA₄, wherein the porphyrin group is pyropheophorbide-a. This teledendrimer is exemplified in the structure below:

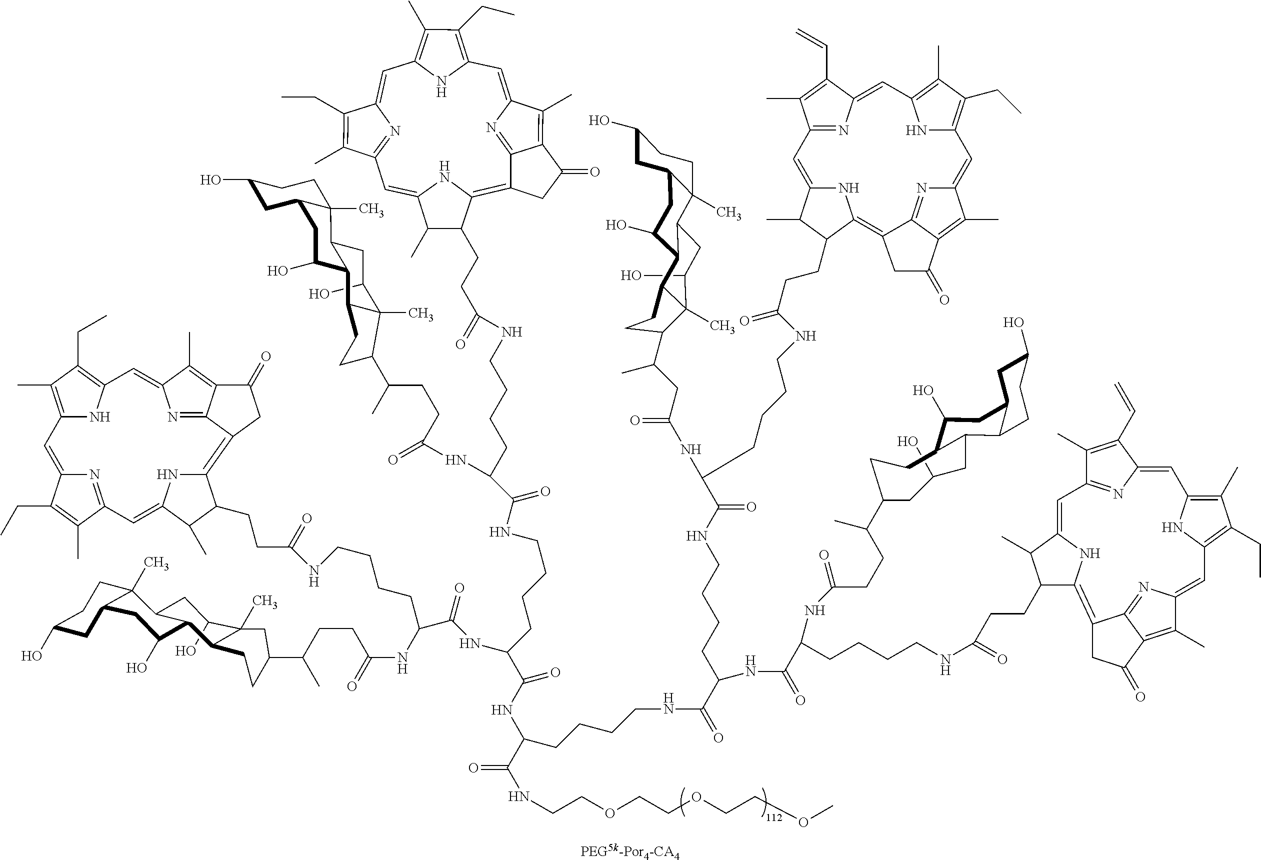

In Vivo Imaging of IH Using NP:

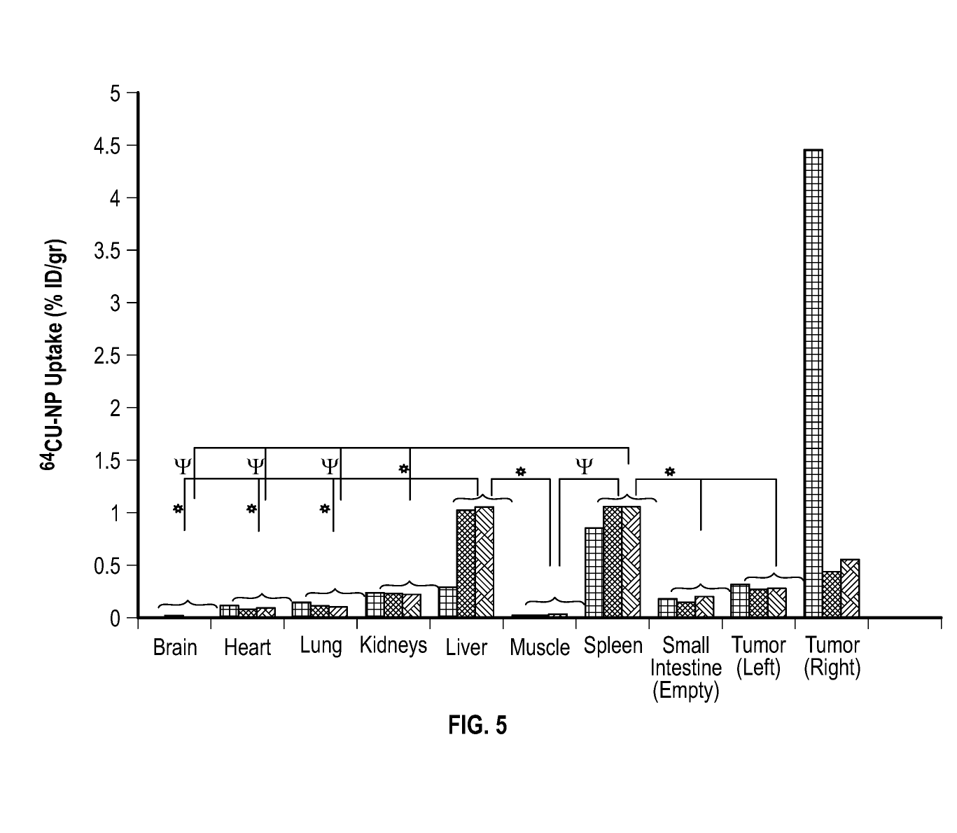

Using PET and NIRF imaging it has been demonstrated that the NP accumulates in IH allografts. For PET imaging, IH bearing nude mice were injected with NP labeled with Copper 64 (⁶⁴Cu—NP) via tail vein and PET images obtained at 3, 6, 24 and 48 hours post-injection (FIG. 4A-4C). IH showed NP uptake as early as 3 hours post-injection with levels remaining high until 6 hours post-injection and uptake detected with PET imaging correlated well with location of IH on CT imaging (FIG. 4B). An ex vivo biodistribution study was performed at 48 hours and results were parallel with those from PET imaging (FIGS. 4C and 5).

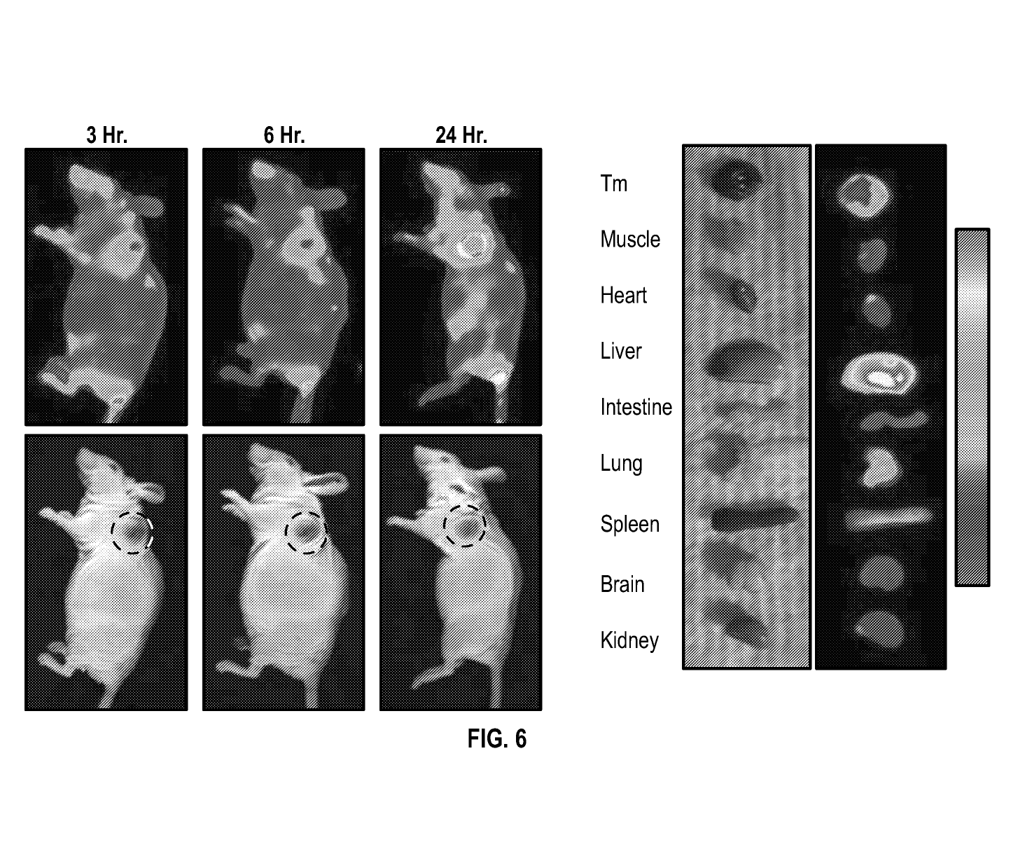

For NIRFI, standard nanomicelle loaded with a hydrophobic fluorophore (DiD) was injected into animals via tail vein and images obtained at 3, 6, and 24 hours post-injection using Kodak image station 4000MM. The accumulated dose of nanomicelles in the IH peaked at 24 hours (FIG. 6). Ex vivo imaging revealed most of the injected dose was taken up by the liver and lung, followed by the IH (FIG. 6). Peak accumulation time is different between PET imaging and NIRFI. This may be explained by the use of NP for PET imaging, and DiD-loaded standard nanomicelle for NIRFI. These nanoparticles are both nanomicelles but have different uptake kinetics because of the minor differences in their chemical structure. Regardless, NP or standard nanomicelle can be used as a nanocarrier. Overall, in vivo data presented here in combination with previous published data demonstrates proof of concept for this proposal and lays the ground for the treatment arm of this study.

Treatment of IH with NP Mediated PDT:

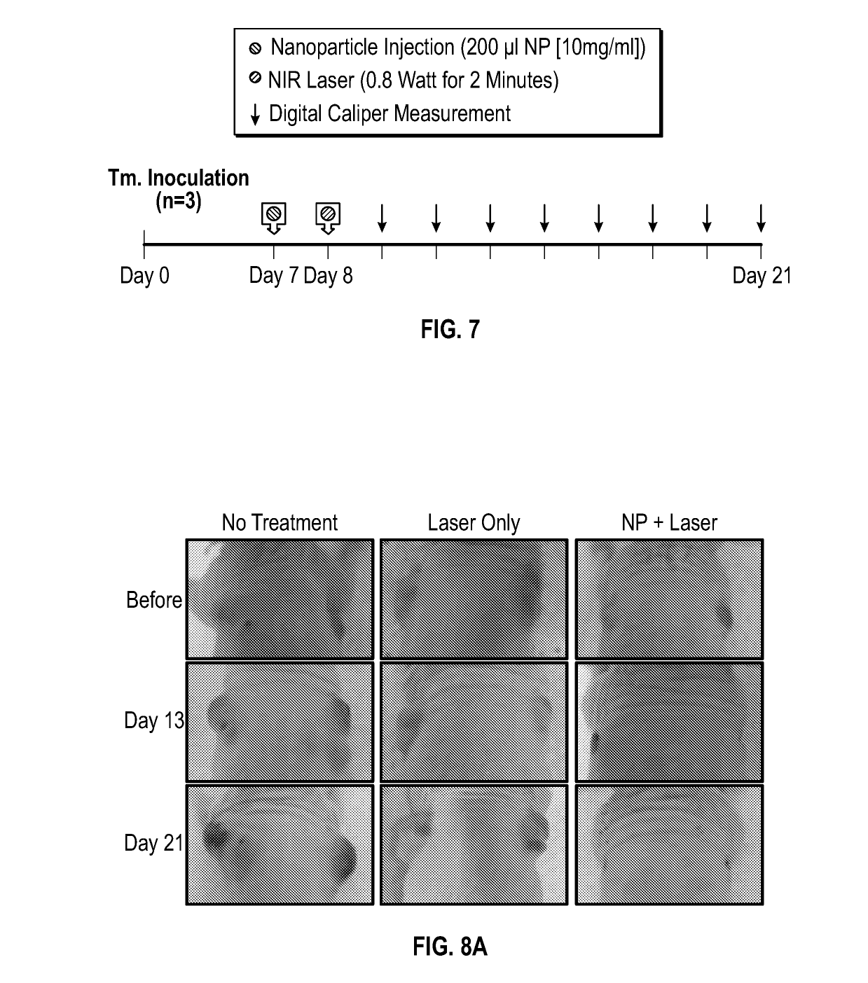

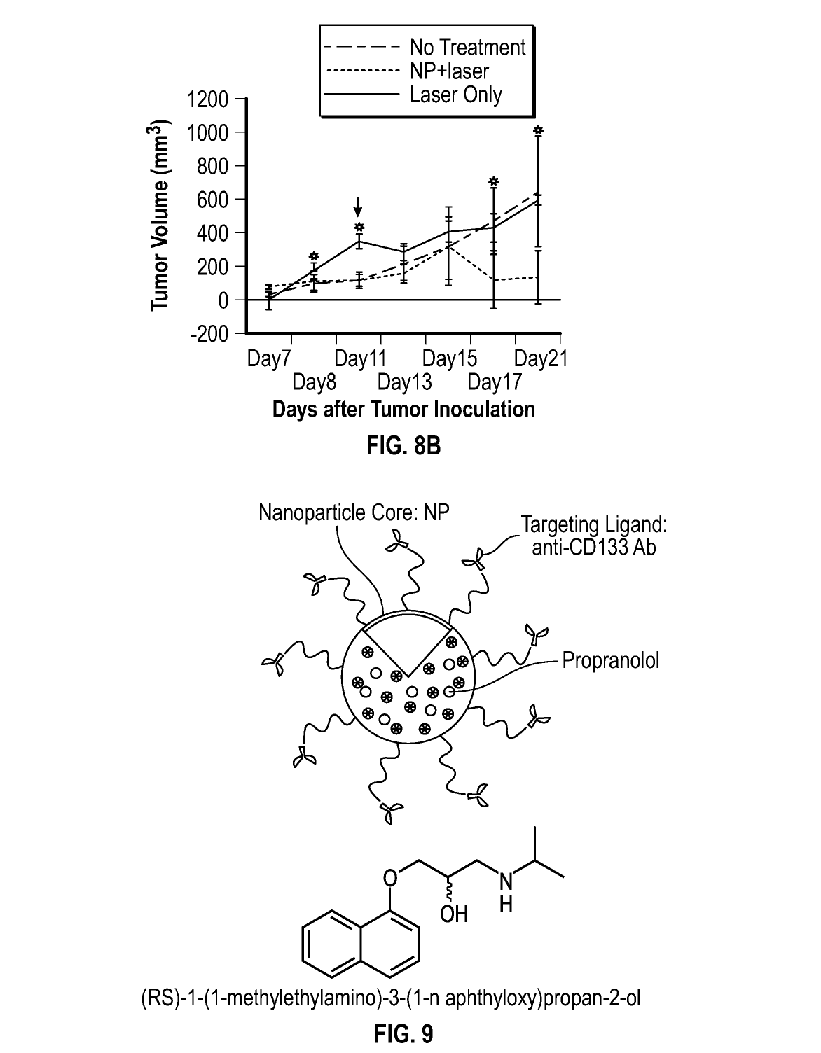

6 animals bearing IH were randomized into 2 groups (n=3 each). The animals in group I were treated with PDT (i.e. NIRL illumination after injection of NP) after tumor inoculation and animals in group II received only PBS injections to serve as controls (FIG. 7). IHs in treatment group regressed soon after the treatment and disappeared totally on day 21 after inoculation and 10 days after treatment (FIG. 8).

Significantly increased NP uptake was observed in the tumor, allowing for a more specific, localized treatment. The treatment with NP and phototherapy stops the growth of IH efficiently. Additional study groups using a vascularization inhibitor (e.g., propanalol) are illustrated in FIG. 9.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of treating a vascular abnormality in a subject in need thereof by photodynamic or photothermal therapy, the method comprising:
a) administering to the subject an effective amount of a photosensitizer; and
b) exposing the vascular abnormality to an effective amount of electromagnetic radiation having a wavelength that is absorbed by the photosensitizer, thereby treating the vascular abnormality by photodynamic or photothermal therapy, wherein the photosensitizer comprises a compound of formula I:
(B)ₖ-(PEG)ₘ-A(Y¹)ₚ-L¹-D-[Y²-L²-R]ₙ (I)
wherein
B is a binding ligand;
each PEG is a polyethyleneglycol (PEG) polymer having a molecular weight of 1-kDa;
A comprises at least one branched monomer unit X and is linked to at least one PEG group;
D is a dendritic polymer having a single focal point group, a plurality of branched monomer units X and a plurality of end groups;
each Y¹ and Y² is absent or a crosslinkable group independently selected from the group consisting of boronic acid, dihydroxybenzene and a thiol;
each L¹ and L² is independently a bond or a linker, wherein L¹ is linked to the focal point group of the dendritic polymer;
each R is independently selected from the group consisting of the end group of the dendritic polymer, a porphyrin, a hydrophobic group, a hydrophilic group, an amphiphilic compound and a drug, wherein at least one R group is a porphyrin;
subscript k is 0 or 1;
subscript m is an integer from 0 to 20;
subscript n is an integer from 2 to 20, wherein subscript n is equal to the number of end groups on the dendritic polymer; and
subscript p is from 0 to 8.

2. The method of claim 1, wherein the vascular abnormality is a vascular tumor.

3. The method of claim 2, wherein the vascular tumor is selected from the group consisting of an hemangioma, a congenital hemangioma, an infantile hemangioma, a tufted angioma, a hemangio endothelioma, a Pyogenous granuloma, and a Kaposiform hamangioendothelioma.

4. The method of claim 3, wherein the hemangioma is an infantile hemangioma.

5. The method of claim 1, wherein the vascular abnormality is a vascular malformation.

6. The method of claim 5, wherein the vascular malformation is a capillary malformation, a venous malformation, a lymphatic malformation, or an arteriovenous malformation.

7. The method of claim 6, wherein the capillary malformation is a port-wine stain.

8. The method of claim 1, wherein the method further comprises administering to the subject an effective amount of an inhibitor of vascularization.

9. The method of claim 8, wherein the inhibitor of vascularization comprises vincristine.

10. The method of claim 8, wherein the inhibitor of vascularization is selected from the group consisting of propranolol, metoprolol, atenolol, acebutolol, nadolol, pindolol, alprenolol, timolol, an inhibitor of ERK, or an inhibitor of VEGFR-2.

11. The method of claim 8, wherein the inhibitor of vascularization is an inhibitor of beta-adrenergic receptor signaling.

12. The method of claim 11, wherein the inhibitor of beta-adrenergic receptor signaling is propranolol, metoprolol, atenolol, acebutolol, nadolol, pindolol, alprenolol, or timolol.

13. The method of claim 11, wherein the inhibitor of beta-adrenergic receptor signaling is an inhibitor of ERK or VEGFR-2.

14. The method of claim 8, wherein the inhibitor of vascularization is administered systemically.

15. The method of claim 8, wherein the inhibitor of vascularization is administered via local injection.

16. The method of claim 8, wherein the inhibitor of vascularization is administered topically.

17. The method of claim 16, wherein the topically administered inhibitor of vascularization is timolol.

18. The method of claim 1, wherein the photosensitizer is administered topically.

19. The method of claim 1, wherein the photosensitizer is administered via local injection.

20. The method of claim 1, wherein the photosensitizer is administered systemically.

21. The method of claim 8, wherein the photosensitizer is a nanocarrier having an interior and an exterior, the nanocarrier comprising a plurality of first conjugates wherein each conjugate comprises:
a polyethylene glycol (PEG) polymer;
at least two amphiphilic compounds having both a hydrophilic face and a hydrophobic face;
at least one porphyrin;
optionally at least two crosslinking groups; and
a dendritic polymer covalently attached to the PEG, the amphiphilic compounds, the porphyrin and the crosslinking groups,
wherein each conjugate self-assembles in an aqueous solvent to form the nanocarrier such that a hydrophobic pocket is formed in the interior of the nanocarrier by the orientation of the hydrophobic face of each amphiphilic compound towards each other, wherein the PEG of each conjugate self-assembles on the exterior of the nanocarrier, and
wherein each conjugate is a compound of formula I:
(B)ₖ-(PEG)ₘ-A(Y¹)ₚ-L¹-D-[Y²-L²-R]ₙ (I)
wherein
B is a binding ligand;
each PEG is a polyethyleneglycol (PEG) polymer having a molecular weight of 1-100 kDa;
A comprises at least one branched monomer unit X and is linked to at least one PEG group;
D is a dendritic polymer having a single focal point group, a plurality of branched monomer units X and a plurality of end groups;
each Y¹ and Y² is absent or a crosslinkable group independently selected from the group consisting of boronic acid, dihydroxybenzene and a thiol;
each L¹ and L² is independently a bond or a linker, wherein L¹ is linked to the focal point group of the dendritic polymer;
each R is independently selected from the group consisting of the end group of the dendritic polymer, a porphyrin, a hydrophobic group, a hydrophilic group, an amphiphilic compound and a drug, wherein at least one R group is a porphyrin;
subscript k is 0 or 1;
subscript m is an integer from 0 to 20;
subscript n is an integer from 2 to 20, wherein subscript n is equal to the number of end groups on the dendritic polymer; and
subscript p is from 0 to 8.

22. The method of claim 21, wherein the nanocarrier further comprises the inhibitor of vascularization, wherein the inhibitor of vascularization is sequestered in the hydrophobic pocket of the nanocarrier.

23. The method of claim 22, wherein the inhibitor of vascularization is an inhibitor of beta-adrenergic receptor signaling, propranolol, metoprolol, atenolol, acebutolol, nadolol, pindolol, alprenolol, timolol, an inhibitor of ERK, an inhibitor of VEGFR-2, or vincristine.

24. The method of claim 21, wherein the nanocarrier is administered topically.

25. The method of claim 21, wherein the nanocarrier is administered by local injection.

26. The method of claim 21, wherein the nanocarrier is administered systemically.

27. The method of claim 22, wherein the nanocarrier is a thiol cross-linked nanocarrier and the exposing the vascular abnormality to an effective amount of electromagnetic radiation breaks the thiol cross-linkages and thereby releases the inhibitor of vascularization.

28. The method of claim 1, wherein the electromagnetic radiation having a wavelength that is absorbed the photosensitizer comprises electromagnetic radiation having a wavelength of about 405 nm or about 680 nm.

29. A composition comprising:
a) a nanocarrier having an interior and an exterior, wherein the interior of the nanocarrier comprises a hydrophobic pocket; and
b) an inhibitor of vascularization, wherein the inhibitor of vascularization is sequestered in the hydrophobic pocket of the nanocarrier,
wherein the nanocarrier comprises a plurality of first conjugates wherein each conjugate comprises:
a polyethylene glycol (PEG) polymer;
at least two amphiphilic compounds having both a hydrophilic face and a hydrophobic face;
at least one porphyrin;
optionally at least two crosslinking groups; and
a dendritic polymer covalently attached to the PEG, the amphiphilic compounds, the porphyrin and the crosslinking groups,
wherein each conjugate self-assembles in an aqueous solvent to form the nanocarrier such that a hydrophobic pocket is formed in the interior of the nanocarrier by the orientation of the hydrophobic face of each amphiphilic compound towards each other, wherein the PEG of each conjugate self-assembles on the exterior of the nanocarrier, and
wherein each conjugate is a compound of formula I:
(B)ₖ-(PEG)ₘ-A(Y¹)ₚ-L¹-D-[Y²-L²-R]ₙ (1)
wherein
B is a binding ligand;
each PEG is a polyethyleneglycol (PEG) polymer having a molecular weight of 1-100 kDa;
A comprises at least one branched monomer unit X and is linked to at least one PEG group;
D is a dendritic polymer having a single focal point group, a plurality of branched monomer units X and a plurality of end groups;
each Y¹ and Y² is absent or a crosslinkable group independently selected from the group consisting of boronic acid, dihydroxybenzene and a thiol;
each L¹ and L² is independently a bond or a linker, wherein L¹ is linked to the focal point group of the dendritic polymer;
each R is independently selected from the group consisting of the end group of the dendritic polymer, a porphyrin, a hydrophobic group, a hydrophilic group, an amphiphilic compound and a drug, wherein at least one R group is a porphyrin;
subscript k is 0 or 1;
subscript m is an integer from 0 to 20;
subscript n is an integer from 2 to 20, wherein subscript n is equal to the number of end groups on the dendritic polymer; and
subscript p is from 0 to 8.

30. The composition of claim 29, wherein the inhibitor of vascularization is an inhibitor of beta-adrenergic receptor signaling, propranolol, metoprolol, atenolol, acebutolol, nadolol, pindolol, alprenolol, timolol, an inhibitor of ERK, an inhibitor of VEGFR-2, or vincristine.
